(19) United States
(12) Patent Application Publication
(10) Pub. No.: US 2023/0162140 A1
(43) Pub. Date: May 25, 2023

(54) FORECAST ENGINE FOR FLEET MANAGEMENT

(71) Applicant: EquipX, LLC, Aventura, FL (US)

(72) Inventors: Ilan Mintz, Aventura, FL (US); Yair Granek, Fairfax, VA (US)

(21) Appl. No.: 17/531,677

(22) Filed: Nov. 19, 2021

(51) Int. Cl. G06Q 10/08; G16H 40/40; G06Q 10/06; G06Q 30/02

(52) U.S. Cl. CPC G06Q 10/087; G16H 40/40; G06Q 10/06393; G06Q 30/0206

(57) ABSTRACT

A forecast engine provides forecast for replacement of equipment in a fleet, particularly beneficial for medical equipment. A database stores parameter data of various medical equipment belonging to the organization. An appraisal module uses the parameter data to generate an appraised value of each of the medical equipment for each year in a forecast period. Upon selection by a user of a specific modality, the forecast engine provides suggestions for equipment replacement annually according to utilization and budget constraints. The entire system may reside in the cloud.

### FIELD

The present disclosure generally relates to fleet forecast engines and, more particularly, to forecast engines for a fleet of medical equipment.

### DESCRIPTION OF THE RELATED ART

Pre-owned or used medical equipment is an often-overlooked asset in a hospital or medical practice. Generally, decisions regarding capital spending on medical equipment are made by consensus of a committee, based on historical trends, rather than forecasting for future needs. The medical equipment of a medical facility is generally counted as an expense, and its value is indicated only by its depreciated value in the accounting ledger, rather than its value in the used market place.

Most health systems do not know the operating costs, revenue, utilization and profitability of their capital equipment. The lack of metrics (e.g., profit and loss) for capital equipment means that clinical service line leaders have no visibility into the contribution they make to the health system's profitability. The depreciated book values of a health system's most expensive equipment typically represent only a fraction of its current market value. Moreover, at the end of the depreciation period, the book value of depreciated equipment is zero, while it may fetch thousands of dollars on the used equipment market.

Moreover, managers have no data with which to make a decision relating to any of the capital equipment in their healthcare system. For example, a specific machine may require much maintenance, which reduces its availability, thus reducing its normalized utilization. Consequently, the equipment may be operating at a loss, without managers having any data to be alerted to such a situation. Additionally, even when the increased maintenance costs are detected, there is no information to a manager to decide which action would provide a better financial return, a refurbishment, an upgrade, a replacement, a lease, etc.

What is needed is a system and method for evaluating medical equipment enabling better evaluation of equipment utilization efficiency and forecast-based purchasing and selling decisions.

### SUMMARY

The following summary of the disclosure is included in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

Applicant has previously disclosed systems designed to help resolving the above-detailed problems, see, e.g., U.S. application Ser. No. 17/000,267, filed Aug. 21, 2020, and U.S. application Ser. No. 17/127,185, filed Dec. 18, 2020, which are incorporated herein in their entirety by reference. Embodiments disclosed herein provide further solutions to assist fleet managers in making decisions regarding fleet of equipment, such as fleet of medical equipment. Similar solutions disclosed herein can be implemented for fleets in other fields, e.g., land, marine and aviation vehicle fleets, etc. To simplify the disclosure and provide concrete examples, the disclosed embodiments relate to fleet of medical equipment managed by an Integrated Delivery Network (IDN), otherwise known as Healthcare System. An IDN may manage equipment operating in its hospitals, clinics (e.g., outpatient clinics), free-standing labs (e.g. imaging centers), etc., each serving a particular population and a particular geographical area.

Embodiments disclosed herein provide financial and operational visibility to a healthcare organization regarding each individual asset employed in the organization. Disclosed embodiments also provide complete transparency into capital equipment performance throughout its entire lifecycle. Disclosed embodiments also provide detailed forecasting of maintenance and replacement of the fleet based on predictive analytics. Moreover, by providing transparency and automated forecast based on defined criteria, better financial decisions regarding the equipment can be made.

Disclosed embodiments provide systems and methods for forecasting need for replacement of medical equipment. Aspects of disclosed embodiments provide solutions for medical systems and hospitals to forecast budgets and ensure available equipment meets the hospital's health care needs.

Disclosed aspects include a computerized method for performing forecast of service equipment in a fleet, the method includes at least the steps of: generating a user interface enabling a user to set a forecast period; obtaining operation data of the service equipment in the fleet; determining an appraised value of each of the service equipment for each year in the forecast period; assigning a score to each of the service equipment for each year of the forecast period; determining utilization rate for each of the service equipment for each year of the forecast period; determining predicted profitability for each of the service equipment for each year of the forecast period; using the score, the appraised value, the predicted profitability and the utilization rate, to generate an equipment replacement forecast by determining for each year of the forecast period whether any of the service equipment should be replaced; and, displaying the equipment replacement forecast on the user interface.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 is a block diagram illustrating a system environment for the forecast engine, according to an embodiment.

FIG. 2 illustrates an example of a screenshot listing various forecast analyses, according to an embodiment.

FIG. 3 illustrates a block chart for various entries that may be used to run a new forecast, according to an embodiment.

FIG. 4 illustrates a screenshot showing user interface with scoring for operations and business categories, according to an embodiment.

FIG. 5 illustrates a screenshot showing user interface with scoring for clinical category, according to an embodiment.

FIG. 6 illustrates an example of a screenshot of the simulated scoring, according to an embodiment.

FIG. 7 illustrates a screenshot of the user interface for the tuning, according to an embodiment.

FIG. 8 is a general schematic illustrating a screenshot that may be used to enter or change variables related to purchasing or leasing new equipment, according to an embodiment.

FIG. 9 illustrates a screenshot of a user interface allowing the user to set adjustments to the EquipX™ value, according to an embodiment.

FIG. 10 illustrates an example of a screenshot for input of predicated demographic changes, according to an embodiment.

FIG. 11 illustrates an example of a screenshot for input of predicated Medicare changes, according to an embodiment.

FIG. 12 illustrates an example of a screenshot for input of predicated medical cost changes, according to an embodiment.

FIG. 13 illustrates an example of a screenshot presenting a summary of the forecast for the specific modality selected, according to an embodiment.

FIG. 14 is an example of an element of a screenshot for a fleet forecast dashboard, according to an embodiment.

FIG. 15 is an example of another element of a screenshot for a fleet forecast dashboard, according to an embodiment.

FIG. 16 is an example of further element of a screenshot for a fleet forecast dashboard, according to an embodiment.

FIG. 17 illustrates an example for traffic light report according to an embodiment.

FIG. 18 illustrates an example for journal report according to an embodiment.

FIG. 19 illustrates another example for journal report according to an embodiment.

FIG. 20 illustrates an example for side by side inventory report according to an embodiment.

FIG. 21 illustrates an example for side by side utilization report according to an embodiment.

FIG. 22 illustrates an example for side by side financial report according to an embodiment.

FIG. 23 schematically shows a block diagram of an apparatus for generating the forecasts according to example implementations of the present disclosure.

### DETAILED DESCRIPTION

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention; however, the order of description should not be construed as to imply that these operations are necessarily order dependent. In particular, these operations need not be performed in the order of presentation. Moreover, different features may be highlighted in different embodiments, but should not be construed as limited only to the embodiment within which they are disclosed. Indeed, the features may be “mixed and matched” with different embodiments, as one finds different benefits.

As indicated above, Applicant previously disclosed systems for supporting fleet managers, and some parts of the embodiments disclosed herein may rely on certain features disclosed in the above-cited Applications. For example, Applicant previously disclosed a system for determining market value for a particular equipment of the fleet, referred to herein as EquipX™ value, which will be made use of in certain aspects of the disclosed embodiments. The reader is highly encouraged to review the cited Applications so as to obtain familiarity of the background for the embodiments disclosed herein, including methods for calculating the EquipX™ value.

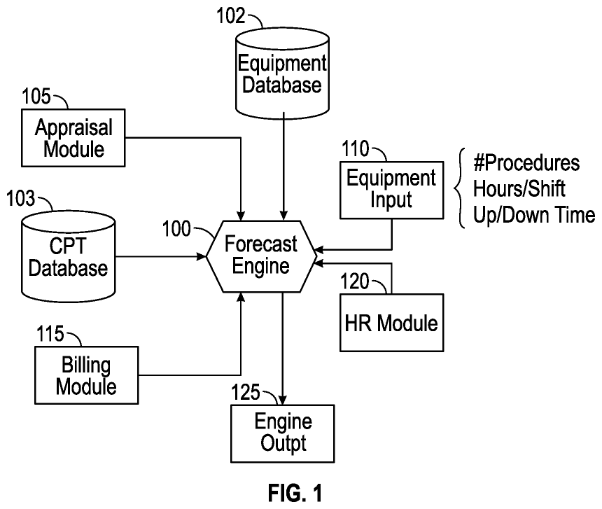

FIG. 1 is a block diagram illustrating a system environment for the forecast engine, according to an embodiment. While FIG. 1 illustrates various modules that may interact with the forecast engine, in other embodiments more or fewer modules may be used, depending on availability and implementation. For example, it is considered that optimal results would be achieved by implementing the forecast engine within the environment of the decision support engine and the evaluation engine disclosed in the above-cited Applications, but adequate results may also be achieved using a “stand alone” implementation.

Additionally, it should also be noted that each module and/or process and combinations of the modules and/or processes disclosed herein can be implemented by a hardware-based system exclusive for executing stipulated functions or actions, or by a combination of dedicated or general purpose hardware and computer instructions. For examples, modules and processes disclosed herein may be implemented as a computer software operating on a general purpose computer or sever, such as, via the Cloud. For example, a user may interact with any of the modules and processes described using a client connected to a server via the Internet or a dedicated intranet.

In the example of FIG. 1, the forecast engine 100 is coupled to and receive information from various modules. For example, appraisal module 105 sends appraised value of currently operating equipment within the IDN. In one example, the appraisal module 105 may be the EquipX™ module disclosed in the above-cited Applications. Also, current procedural terminology (CPT) database module 103 may be coupled to the appraisal module 105, to the forecast engine 100, or to both, via communication network or bus. The CPT database 103 code-set lists medical, surgical, and diagnostic services and is designed to communicate uniform information about medical services and procedures among physicians, coders, patients, accreditation organizations, and payers for administrative, financial, and analytical purposes. This CPT database is updated periodically according to publications by the American Medical Association. The CPT database 103 also stores the relative value units (RVUs), which are a measure of value used in the United States Medicare reimbursement formula for physician services. As far as cost per procedure, the forecast engine can use the RVU's, Medicare reimbursement amount, or actual prior data e.g., from billing module 115. Also, the forecast engine may fetch the CPT codes corresponding to new procedures made possible using newly upgraded or purchased equipment. Using these new CPT codes, the forecast engine 100 may calculate potential increase in utilization and provide a new procedure volume prediction. Similarly, the appraisal module 105 can use the CPT code to update the appraised value of the upgraded or replaced equipment.

Data regarding currently operational equipment may be provided to the forecast engine 100 from an equipment database 102, if it exists, or from the individual equipment directly, as equipment input 110. The equipment input 110 may include the number of procedures performed per day or months, operational hours per shift, up time, down time, etc. The data from the HR module 120 may be used to estimate labor costs of operating the equipment. HR module 120 may refer to a Human Resources database, a payroll database, or any other labor cost database.

The forecast engine output 125 is provided and displayed on a monitor, and can be edited and modified in real time, or it can be saved for further work at a later time. The system provides the user with the ability to save any forecast run, so that at a future date the user may return to an already run forecast and review or perhaps rerun it with updated data or different variables. Thus, the system provides a list of previously run forecast by all registered users, so that any user may start a new forecast or open an already run forecast and use that as a starting point.

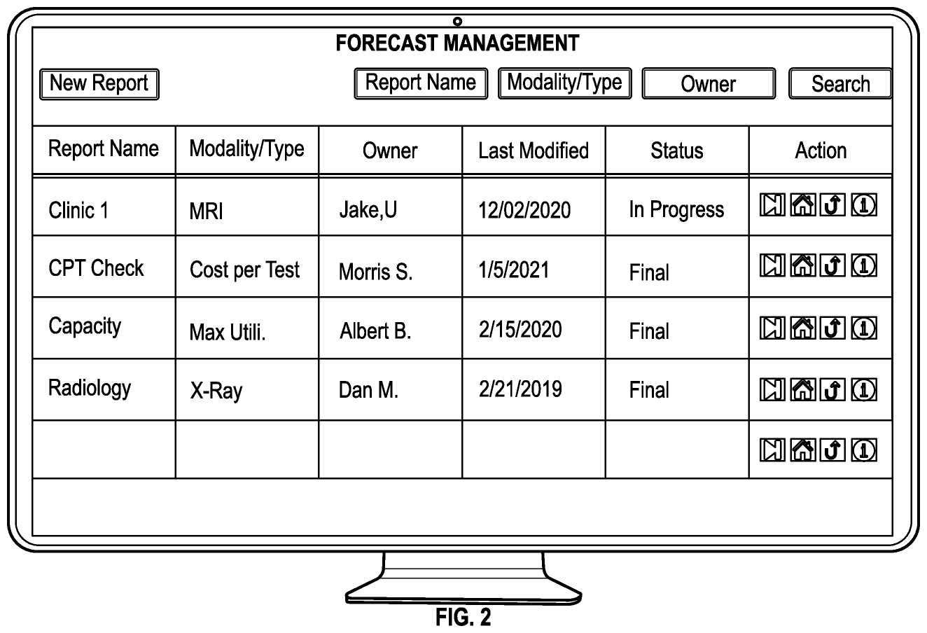

FIG. 2 illustrates an example of a screenshot listing various reports, including forecast analyses, previously performed and saved by various users in the organization. In the example of FIG. 2, the forecast model is run within a complete management system, such as that disclosed in the above-cited application, so the list includes other types of reports, not just forecast reports. Any user with an approved access may review any of the listed reports and/or forecasts, edit or rerun any of the reports or forecasts using the same or different variables, or run and save a new forecast.

Generally, a healthcare system may divide its operations into regions and particular hospitals/clinics within each region, the hospitals may be divided into wards or departments, and particular equipment may be classified according to a particular modality under certain department. For example, an MRI machine would be classified as one modality in the imaging department. Within the modality there may be specific equipment specialties, such as CT scan 64 slice, MRI 1.5 T, etc. As will be further detailed below, fleet management and forecasting is particularly beneficial when performed on a per modality approach. For example, an MRI modality may provide insights into the future needs of the imaging department and help establish budget for that department. To generalize, modality may be comparable to vehicle type. For example, in land vehicle fleet forecasting, modality or vehicle type may refer to vans, trucks, tractor trailers, etc., while specialty within the trucks modality may be, e.g., flat-bed truck, enclosed truck, moving truck, etc.

In FIG. 2 the top line includes a New Report button, enabling a user to run a new report or forecast. Also, three entry windows are available for the user to enter a report name, modality or type of report, and owner name, and the user may then click “Search” to search the reports according to the entries. The main display includes listing of previously run reports, with entries such as report name, modality/type of report, owner of the report, date the report was last modified, status of the report, and action icons for actions such as exiting the report page, editing the report, deleting the report, etc.

While the forecasting tools described herein may be utilized according to various different views, one particularly interesting approach is to run the forecast from the point of view of corporate performance by modality. This approach provides the current situation of a particular modality across all of the facilities of the IDN. For example, if the modality chosen is MRI, the system would present the current situation of the currently existing MRI machines within the IDN. Operating the forecast engine would then provide a beneficial comparison of current performance and fleet status and target date performance and fleet status.

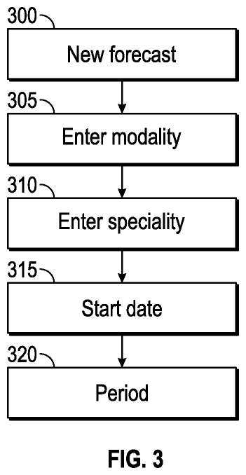

For example, FIG. 3 illustrates a block chart for various entries that may be used to run a new forecast. In FIG. 3, a process for running a new forecast starts at step 300. The user may enter a modality for the new forecast at 305, and optionally at 310 also enter a specific specialty for the modality. At 315 the user enters a start date, which may be any date in the past when data for current status of the modality exists. Then at 320 the user may select the period for running the report, e.g., 5 years, 10 years, etc. When running the forecast, the process would initialize the forecast using the data of the start date, and then recursively process it for each year of the indicated period, e.g., for ten years. To calculate the various parameters of the forecast, the system uses variables, some of which may be fixed in the system and some of which may be available to the user for adjustments, as will be described further below.

In running the forecast, the system utilizes a concept referred to herein as scoring. In disclosed embodiment scoring is generally available for three categories: operations, business and clinical. The operations score relates to the operability and serviceability of the equipment, the business score relates to the financial performance of the equipment, and the clinical score relates to the technological age or capability of the equipment. Each of these categories may have several score entries that may be changed by the user, if desired. The scoring enables grading each equipment in service to generate the forecast report while providing proposed dates for retiring specific equipment according to the scores assigned to the particular equipment. In specific examples, the highest the score assigned to a specific equipment, the further it is advanced in the queue for replacement. Also, in some embodiments a minimum score may be set below which an equipment will not be replaced.

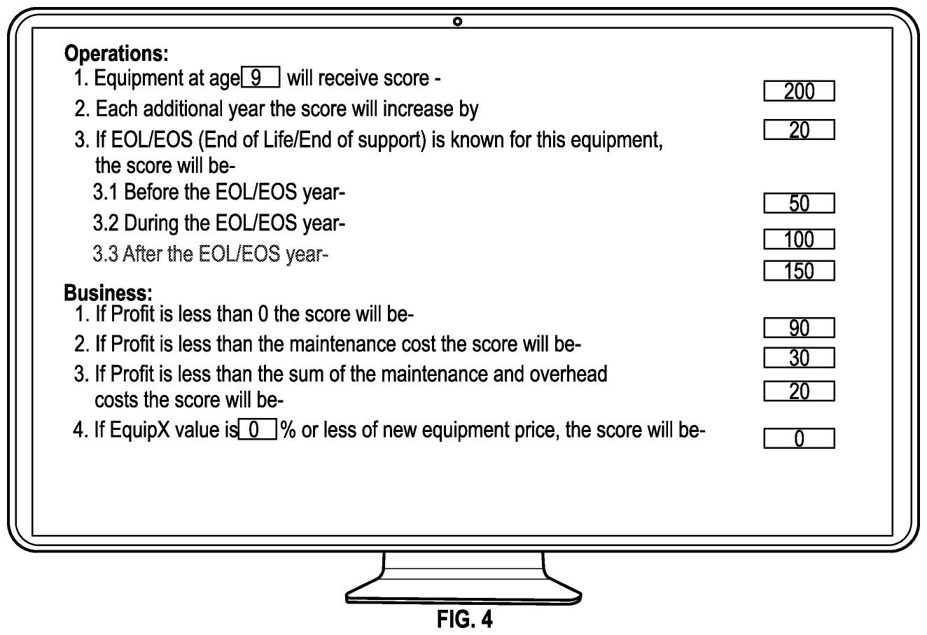

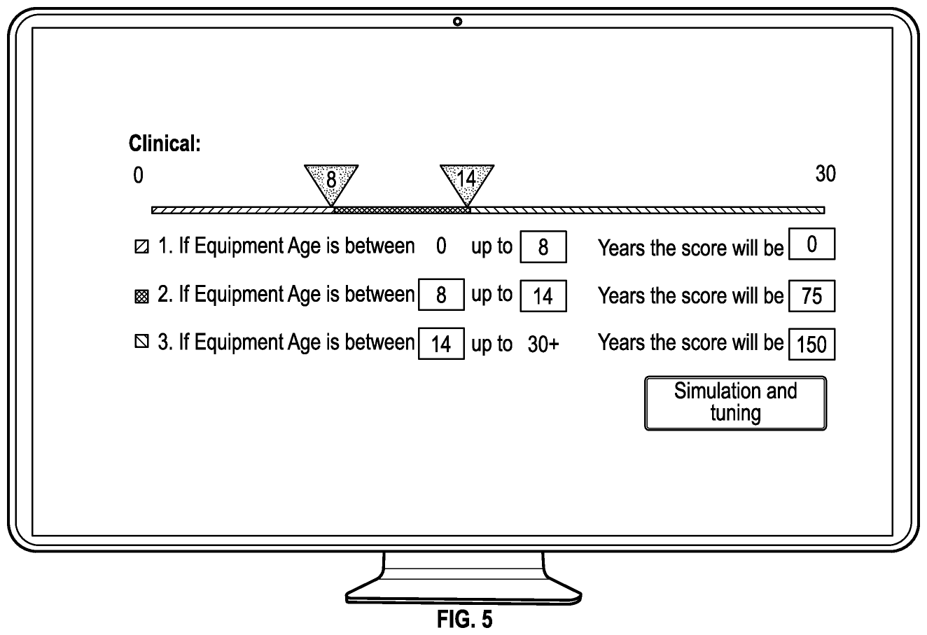

FIG. 4 illustrates a screenshot showing user interface for setting scoring parameters for operations and business categories, while FIG. 5 illustrates a screenshot showing user interface with scoring parameters for the clinical category. In both FIGS. 4 and 5 the entries may be populated by system suggested values, but the user may change each of the values as the user sees fit for a particular situation or modality. The proposed values may incorporate knowledge gained from various entities running the system, including other IDNs, such that these proposed values become more accurate and relevant as more users employ the system. In that sense, the proposed values incorporate in the system form a knowledge-based benchmark for the user.

The scoring for operations generally represent the age of the particular equipment, specifically from the perspective of operability, maintenance costs and reliability. The scoring in this category begins with entering a threshold age, below which the equipment is considered reliable and with normal maintenance costs. For example, a particular equipment may be considered fully reliable up to a specified age, say 10 years, below which no score will be added and above which it will receive a given score, say 200 points. For each additional year over the specified threshold year the score may be increased by a set amount, say 20 or 50 points. Also, if the date for EOL/EOS (End of Life, End of Support) is known for the equipment, a specified score would be added for equipment in service prior to that date, equipment in service during that year, and equipment at service after the date of EOL/EOS. The score may be incremental according to the age of the equipment with respect to the EPL/EOS date. If the manufacturer has not yet announced a date for EOL/EOS, no score is assigned.

The business category generally represents the equipment's financial contribution for the profitability of the entity. The profitability may also relate to various expenditures for the particular equipment. Thus, if the equipment profits for the year is negative (i.e., loss), a specified high scope will be added. If the profitability is less than the maintenance costs for the particular equipment, a lower specified score is added, and if the profitability is less than the maintenance and overhead costs a further lower score is applied. A further scoring may be added for indication that the estimated value of the equipment in service is a certain percentage of a new replacement equipment price.

The clinical category is also tied to the age of the equipment, but is viewed more from the perspective of its technology and performance capability. That is, the older the equipment is, the more likely it lacks features and capabilities of a more modern equipment. For example, some old equipment may be unable to perform various operations that are available on a modern equivalent. Also, newer equipment may be more sensitive, thus providing better imaging, while using less radiation. For the clinical category a user may enter two age values that generally reflect life cycle of the technology of the specific modality selected. For example, for certain modality it may be regarded that up to a certain age, say 8 years, the equipment is considered “current generation” and no additional score is added. Conversely, beyond a certain age, e.g., 20 years, it may be regarded that the equipment is several generations of technologies old and should be imminently replaced, and thus a high score would be applied. In between these two date, a moderate score may be applied as the equipment may not be of current generation, but is functioning sufficiently well to merit a moderate score. Of course, for different modalities the cutoff age would be different according to the relevant technology and life cycle. Therefore, as illustrated in FIG. 5, a slider graphic is provided to enable the user to change the cutoff as necessary.

Thus, in general terms, the scoring module assigns the score for each of the medical equipment within the selected modality and for each year of the forecast period by: assigning points based on predicted operability of each of the medical equipment for that year; assigning points based on predicted profitability of each of the medical equipment for that year; assigning points based on the predicted technology refresh of each of the medical equipment for that year; and, summing up all assigned points to generate the score. The summing may be a straight addition, a weighted addition, or any suitable summing operation.

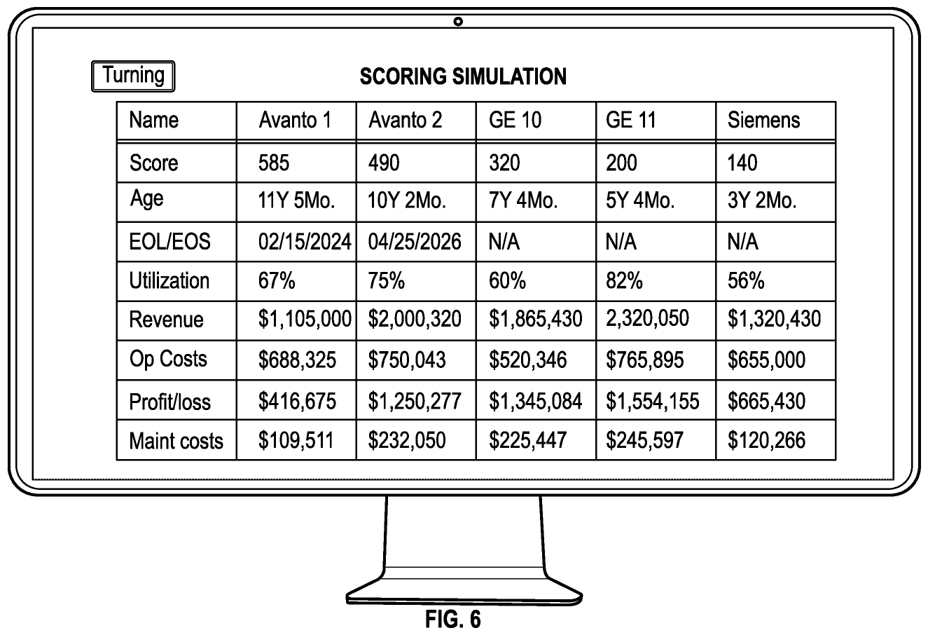

As shown in FIG. 5, the user interface includes a clickable button for performing simulation and enabling tuning of the scoring. The simulation is made available in order to assist the user in understanding the operation of the scoring and the effect of different variables on the scoring of the various equipment under management of the user. Upon clicking the simulation and tuning button, the user is presented with a list of all of the equipment under management of the IDN. The user may then select all the equipment that the user would like to run the simulation on. Once selected, the user may run the simulation, whereupon the user is presented with the scoring of each of the selected equipment. An example of a screenshot of the simulated scoring is illustrated in FIG. 6.

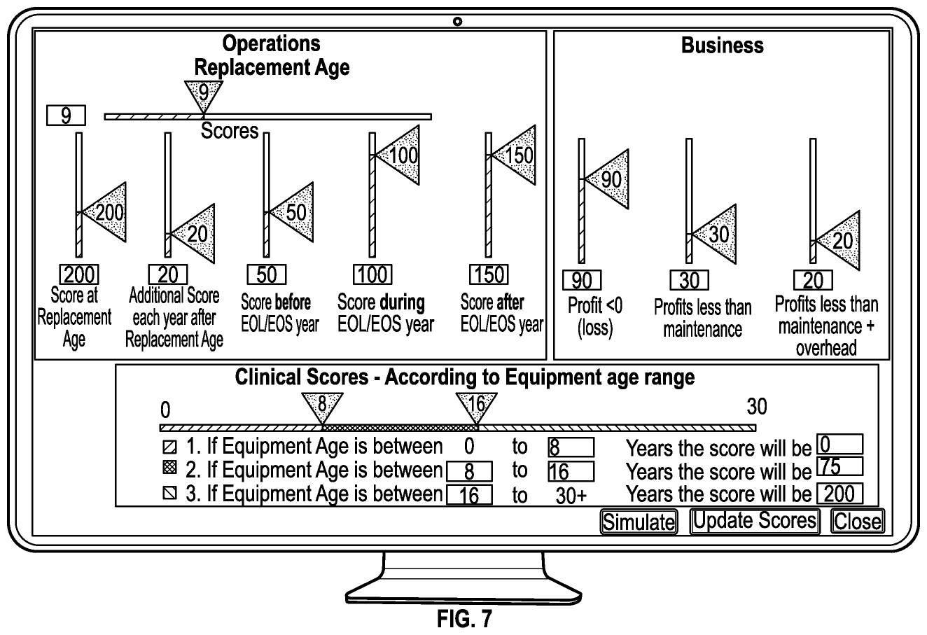

The screenshot of FIG. 6 also includes a “tuning” button, which the user can click to tune the variables of the scoring and see how it affects the simulation. A screenshot of the user interface for the tuning is illustrated in FIG. 7. As can be seen in FIG. 7, various sliders are provided corresponding to the variables shown and discussed with respect to FIGS. 4 and 5, such that the user can drag each slider to change the variable value and then see how it affects the simulation of FIG. 6.

Another consideration for the forecasting is utilization. Generally, utilization can be considered objectively, i.e., according to the maximum utilization capacity of a given equipment, or subjectively, i.e., a normalized utilization according to the particulars of the organization. For example, normalized utilization would consider whether the equipment is operational during the weekend, how many shifts per day the equipment is used, etc. For either the objective or subjective utilization, a user can set a range within which the user would like the utilization to be. For example, the user may set a value for minimum utilization, below which is an indication that the organization has over capacity and perhaps should sell or transfer some equipment to other facility experiencing under capacity. The user may also set a value for maximum utilization, above which it would indicate that the organization may need to acquire additional equipment to service the needs of the organization. Additionally, the user may set a value for minimal equipment per facility. For example, the user may set the value for MRI to 1 per hospital, indicating that each hospital should have at least one MRI machine regardless of actual utilization. Therefore, even if the utilization falls below the minimum utilization, but there's only 1 MRI in that hospital, the forecast results cannot indicate the equipment to be sold or transferred.

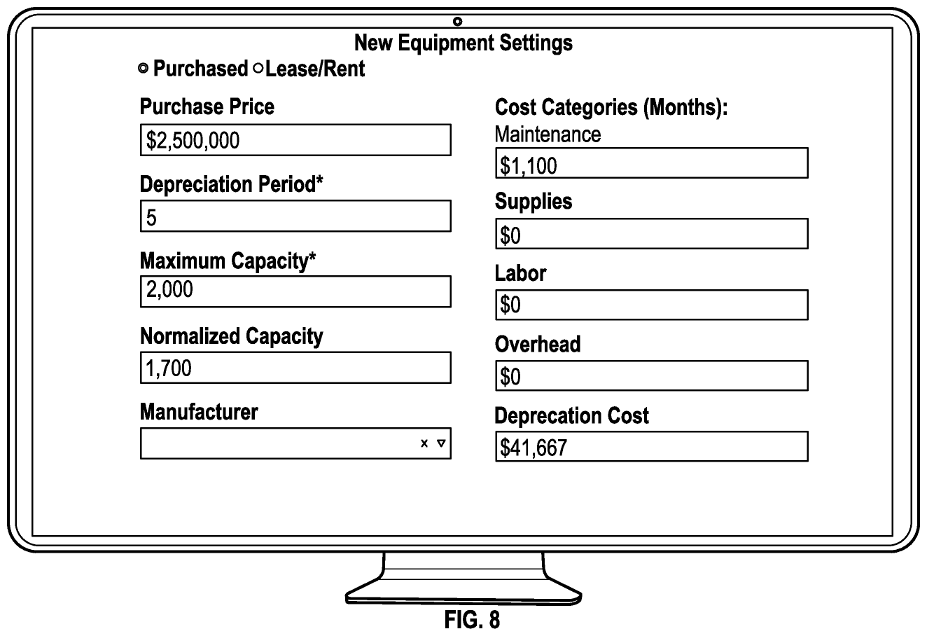

As noted, the system provides forecast for equipment needs in the future. Additionally, the system may also provide recommendations with respect to replacement or new acquisition of equipment. For example, the system can take into account variables relating to purchasing or leasing new equipment. FIG. 8 illustrates a screenshot that may be used to enter or change variables related to purchasing or leasing new equipment. Of course, since the purchasing/lease forecasting is for a time in the future, much of these variables would be estimates, since prices and costs of these items is not certain at the present, and can only be estimated for the future. As shown in FIG. 8, the variables may include purchase price (or monthly lease payments), depreciation period and cost, maximum and normalized capacity, various costs associated with operating the equipment, etc.

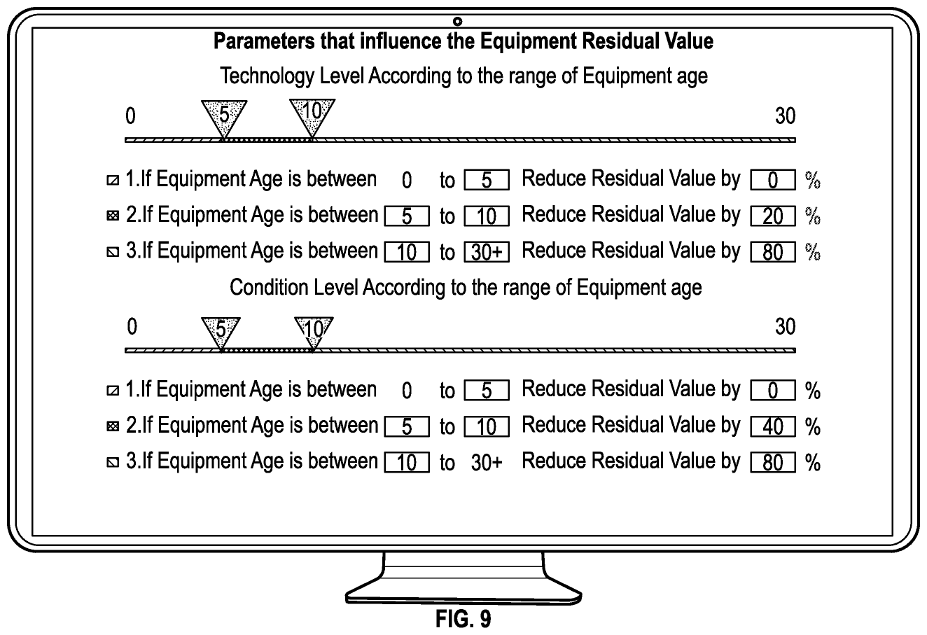

As explained more fully in the above-cited patent applications, especially with respect to estimation of EquipX™ values, the IDN's accounting system would normally depreciate the equipment according to a specific depreciation methodology that was approved by the taxing authorities. Consequently, the “book value” as recorded by the accounting department normally has no relevance to the actual market value of the equipment. This is why the EquipX™ value is such a beneficiary tool for the IDN decision makers. Therefore, the forecast engine also utilizes the EquipX™ value as the proper residual value of the equipment. Generally, the EquipX™ value is estimating the market value if the equipment was to be sold on the day of the calculation of the EquipX™ value. However, the forecast engine also provides suggestions for selling certain equipment in the future. Therefore, for the forecasting engine, the EquipX™ value needs to be adjusted according to the time of the proposed sale. This is illustrated in FIG. 9.

FIG. 9 illustrates a screenshot of a user interface allowing the user to set adjustments to the EquipX™ value calculations. As explained in the above-cited applications, the EquipX™ value estimator utilizes data regarding various factors, assigns weights to each factor, and calculate a market price therefrom. The factors may include historical sales prices, the new equipment price, the age of the equipment, the condition of the equipment (as selected by the user), the status of the equipment (operational, in storage, undergoing refurbishment, etc.), additional parts components and/or upgrades conveyed with the equipment, technological status of the equipment, maintenance costs, disposition costs, and risk factors. The values considered by the estimator are values that are current at the time of the calculations, but the forecaster may indicate a potential sale of an equipment at a time in the future. Therefore, the resulting value estimate may need to be adjusted.

In the example of FIG. 9, the user may decide that if at the future date of sale the equipment is between 1-5 years old, no adjustment will be applied to the EquipX™ value calculations. Conversely, if the equipment at time of sale is older than 5 years, the EquipX™ value calculation will be modified in two specific parameters: technology level and condition level. Thus, depending on the age of the equipment, these two parameters reduced by a given percentage amount, as can be modified by the user. In the example illustrated, if at the time of the forecasted sale the equipment will be between 5-10 years old, the technology level parameter will be reduced by 20% and the condition level parameter will be reduced by 40%. Conversely, if at the time of sale the equipment will be over 10 years old, the technology and condition parameters will be reduced by 80%.

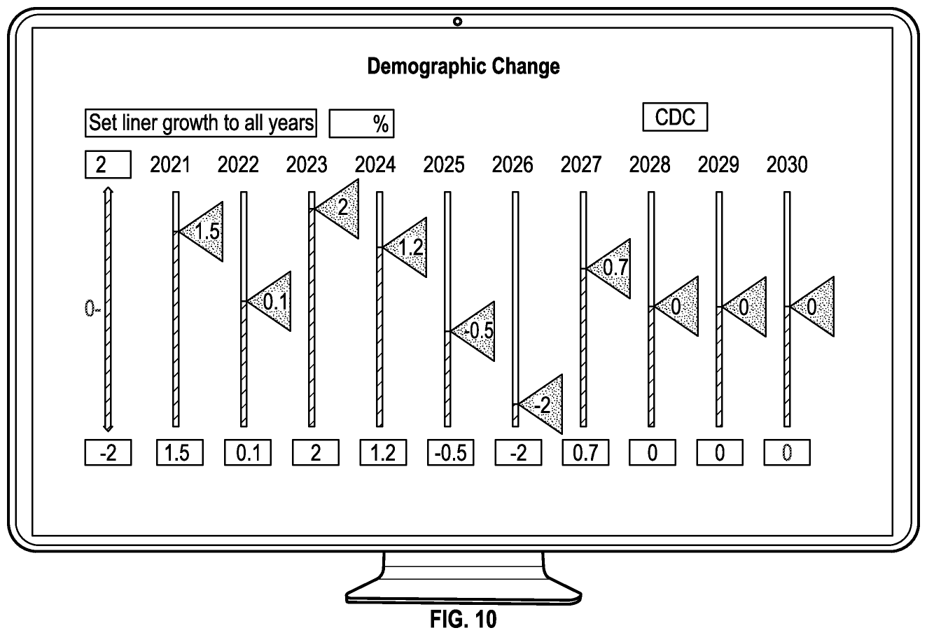

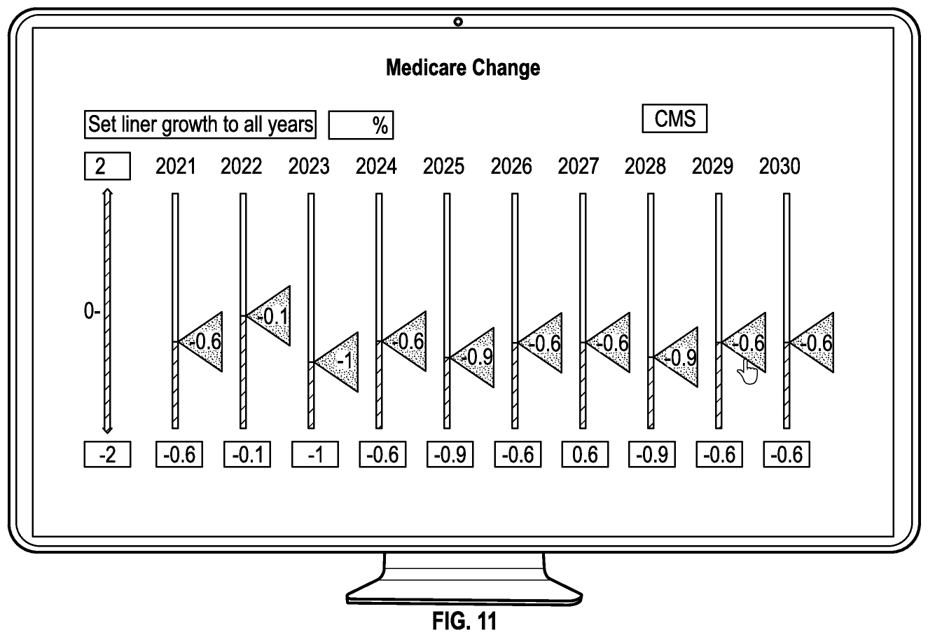

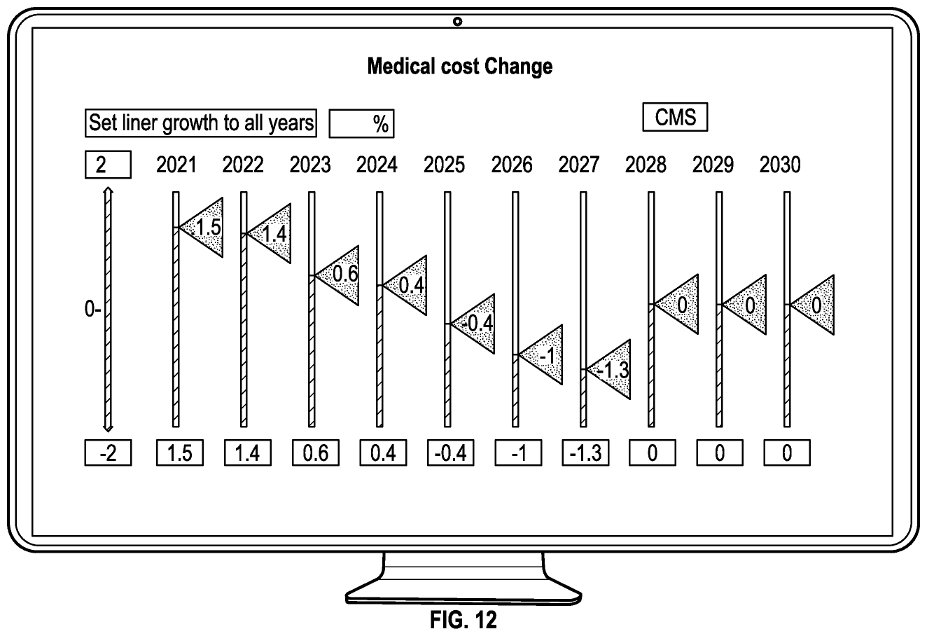

In addition to internal conditions that may affect the forecast, the system can also account for external conditions that are not controlled by the organization, but which may be somewhat predictable. FIGS. 10-12 illustrate examples of screenshots that enable the user to adjust various external parameters. For example, FIG. 10 provides ability to input predicated demographic changes (akin to changes in demand side of the market), in this example, annually for ten years. The user may set a linear growth rate for all of the years, e.g., assume that each year demographics will grow 1% annually. Conversely, the user may decide annual growth/contraction independently for each year by adjusting the sliders. For convenience, a link is included to the CDC website that provides estimates for future demographic changes. An example of data obtainable from the CDC for population projection can be seen in: https://wonder.cdc.gov/population-projections.html. The data can be obtained and sorted by groupings such as, e.g., age, gender, state, projected years, etc. The user can restrict the data to specific states or regions that the organization operates in.

Another external conditions that may affect the forecast are changes in Medicare (which may be comparable to using changes in Consumer Price Index). This is important not just for Medicare reimbursement, since many agreements with various health insurance organizations are based on the Medicare amount as the basis. For example, an agreement with one insurance organization may specify that the insurance would pay for an MRI an amount that is 155% of the amount that Medicare would pay. Since the Medicare payment forms the basis for other payers, forecasting the annual increase in Medicare payments would also account for other insurers' payments. The user is given the ability to either enter a linear annual change or enter specific change for each year. The user is also given a link to the Center for Medicare & Medicaid Services (CMS) to obtain relevant data. An example of data obtainable from the CMS for Medicare projection can be seen in: https://www.cms.gov/Research-Statistics-Data-and-Systems/Research-Statistics-Data-and-Systems.

Also important is the forecasted annual change in the cost for running the equipment, which would also relate to labor costs associated with the equipment. Annual cost estimates can also be obtained from the CMS website. An example of data obtainable from the CMS for Medicare projection can be seen in: https://www.cms/gov/Medicare/Medicare-Fee-for-Service-Payment/PhysicianFeeSched. Another example that can help forecast inflation is https://smartasset.com/investing/inflation-calculator.

In addition to all of the parameters noted above, the user may also indicate certain constraints or thresholds for the forecast. For example, the user may enter a score threshold, below which an equipment should not be in consideration for replacement. The user may also enter total budget for the entire forecast and maximum budget per year for the forecast. Thus, for example, if the user enters $2 million annually, the forecast would not issue a recommendation for replacing equipment at any year that exceeds $2 million. Conversely, the user may set the number of equipment that may be replaced annually or throughout the entire forecast period.

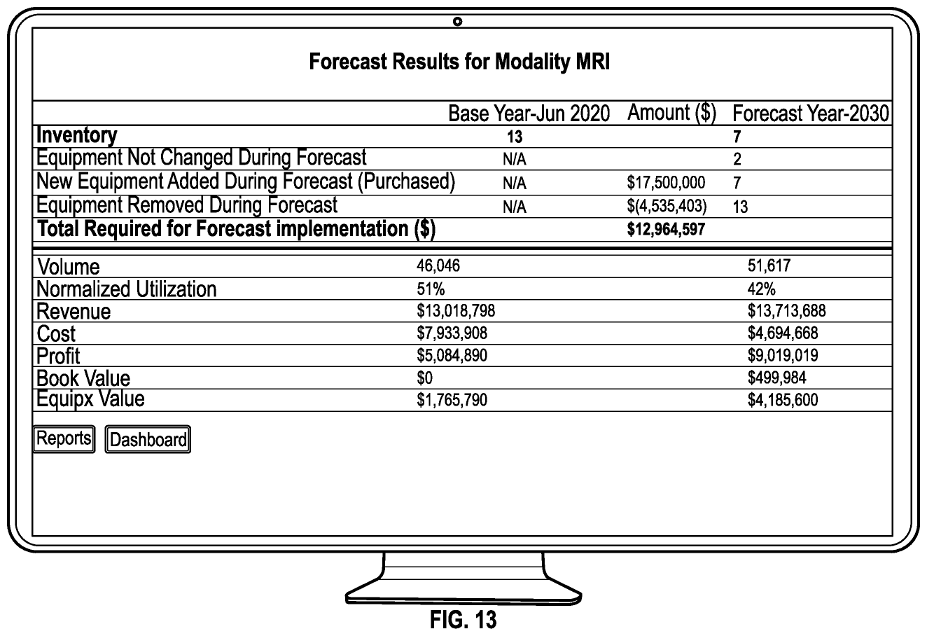

Once the forecast program is run, the user receives a summary of the forecast for the specific modality selected, as illustrated in FIG. 13. In this example the forecast was run for ten years and the summary provides the initial status and the status at the end of the ten-year period. The status includes the number of equipment, the number of equipment that were not changed, the number of equipment that was added, and the number of equipment that was removed, during the ten-year period. Also included in the summary is the cost of the new equipment, the amount of money expected to be received on sell of older equipment, and the net expenditure on new equipment. Importantly, the summary includes a comparison for the initial and ending values of: volume of procedures performed, normalized utilization rate, revenue from the equipment, cost of running the equipment, profits, book value and EquipX™ value. To obtain a more detailed analysis, the interface is provided with two buttons: Reports and Dashboard.

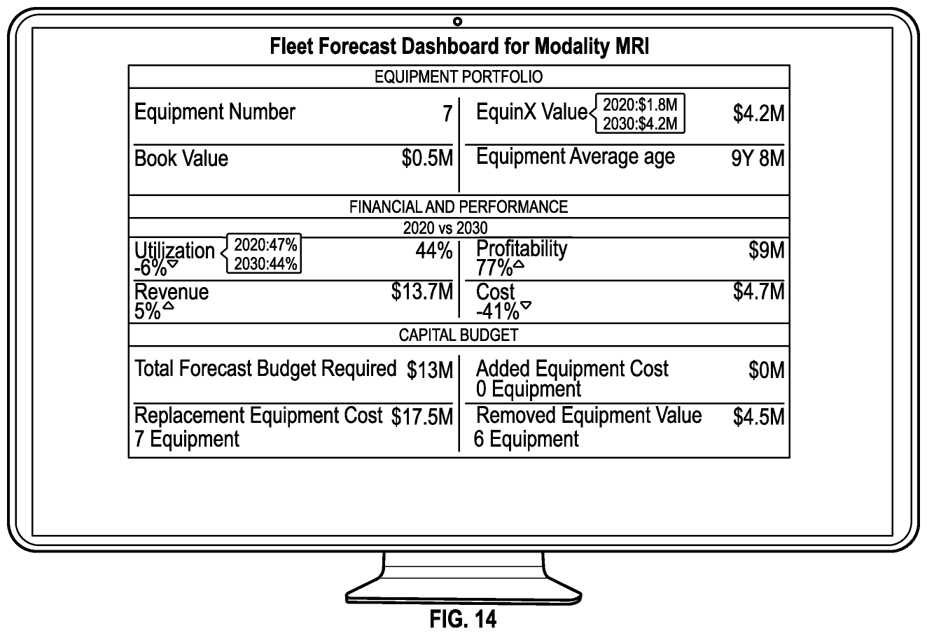

The dashboard is meant to be presented in one screen for ease of viewing, but due to limitations of the drawings' size, the elements of the dashboard are presented in FIGS. 14-16. The values displayed in the dashboard of FIG. 14 are for the status at the end of the forecast period; however, as exemplified in FIGS. 14-16, with mouse over the user can also see more detailed values, e.g., a comparison between the value at the initial date of the forecast and end value for each of the entries. Note that there can be only one mouse over in each screen, but for illustration purposes some of the drawings may have multiple mouse overs.

The first table in FIG. 14 shows the status of the equipment portfolio at the end of the forecast period. As illustrated in this example, at the end of the forecast period, the organization would have seven Mill machines, having (depreciated) book value of $0.5 million and EquipX™ value of $4.2 million (mouse over shows that the initial EquipX™ value was $1.8 million), with average age of 9 years and 8 months. The second table in FIG. 14 shows the financial and performance status at the end of the forecast period. As shown the normalized utilization has dropped slightly to 44% (mouse over shows that it was 47%), but profitability increased drastically to $9 million. This is at least partially due to the savings in the operation costs that was reduced by 41% and increase in revenue by 5%. The third table shows the capital budget for the forecast period. As shown, the total budget required to implement the forecast is $13 million (which, as explained above, can be set as a constraint by the user prior to running the forecast), the cost of purchasing the replacement equipment is $17.5 million, but it is reduced by the $4.5 million received for the sale of the old equipment. Also shown is the cost of any additionally added equipment (i.e., equipment purchased to increase capacity and not as replacement for old equipment). In this example, no equipment is forecasted to be added since the added capacity in this forecast resulted from replacing to new and more capable equipment than the old equipment.

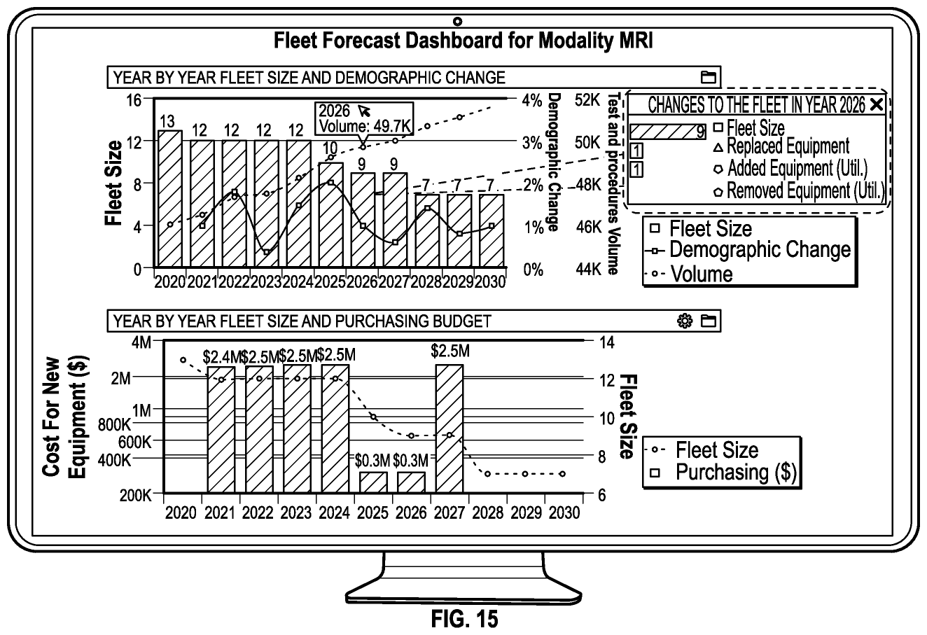

FIG. 15 illustrates two graphs provided in the dashboard: an annual fleet size plotted over demographic changes and an annual fleet size plotted over the purchasing budget. In the first plot, the x-axis represents the year, while there are three y-axes: fleet size, demographic change, and test and procedure volume. The bar plot shows the fleet size for each year, the dot plot shows the annual demographic change, and the diamond plot shows the volume. Using a mouse over, a pop-up is shown with data relevant to the position of the mouse—in the example shown for forecast year 2026 a volume of 49.7 k. Additionally, as illustrated by the callout, by clicking on a bar further data is presented, here the fleet size for that year, and the number of equipment that was replaced, added and removed that year. Note that the demographic change is year by year, while the volume is total procedures per year, so while on average demographic change seems to remain flat, in actuality the plot shows an average growth of about 1% per year over the forecast period. Therefore, the cumulative volume increases over the entire forecast period. Conversely, since old equipment has been replaced with newer and more capable equipment, the fleet size has been reduced, but yet is was capable of providing higher procedures volume.

The second plot in FIG. 15 illustrates the fleet size plotted over capital spending on an annual basis. The bar plot shows the annual capital expenditures (which in this example was capped by the user at $2.5 million), while the dot plot shows the annual fleet size.

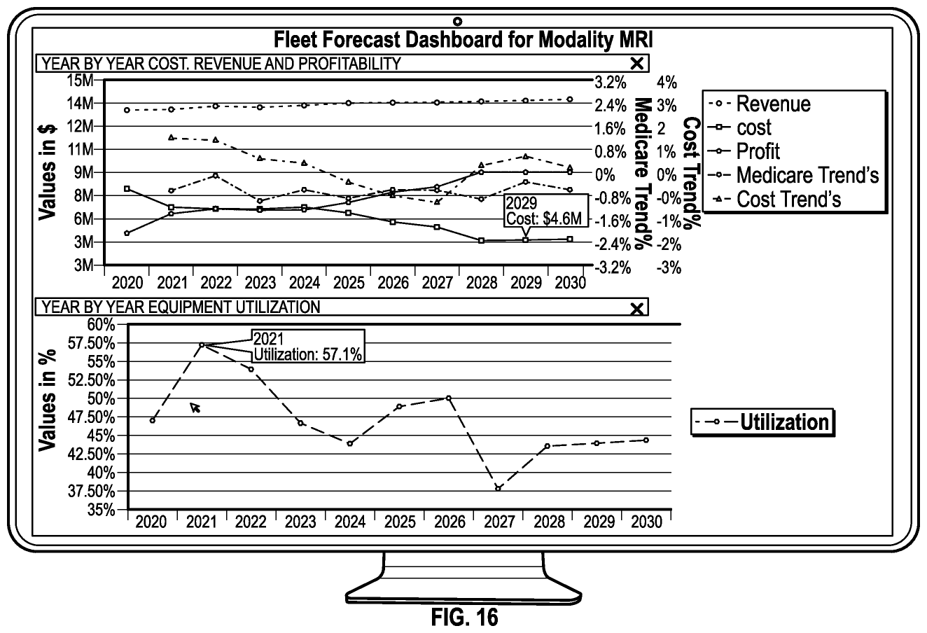

FIG. 16 illustrates additional two graphs provided in the dashboard. The top plot shows the annual cost, revenue, and profitability, while the bottom plot shows the annual fleet utilization. In the top plot, the dots plot indicates the revenue, the diamond indicates cost, the squares indicate profits, the triangle indicates Medicare trend (%), and the inverted triangle indicates cost trend (%).

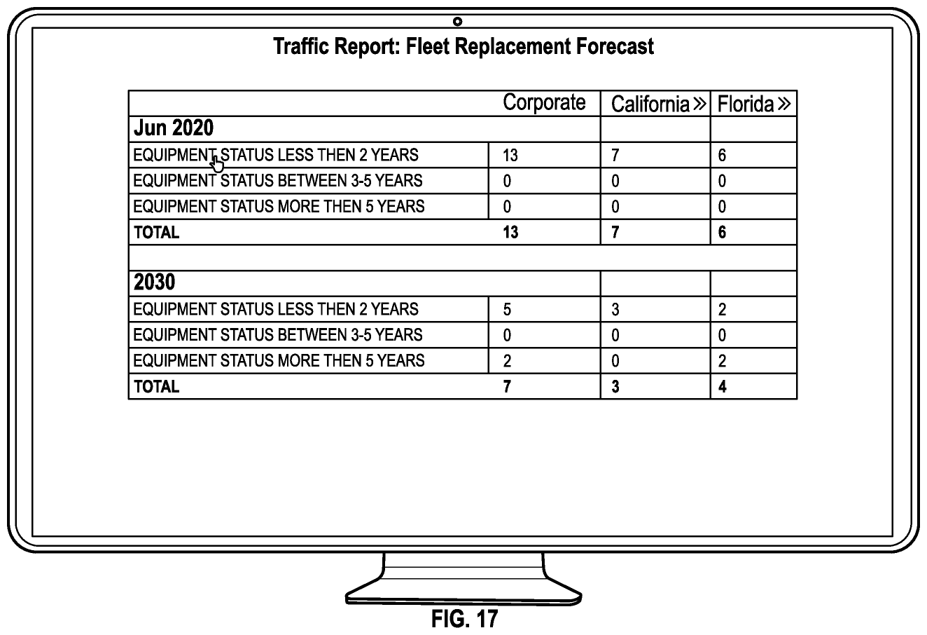

In addition to the dashboard, the user may also select to run several different reports. As shown by the callout in FIG. 13, the reports may include a traffic light report, forecast journal report, side by side performance report, and fleet forecast review report. FIG. 17 illustrates an example of a traffic light report which indicates the status of the equipment at the initial date and at the forecast termination date. For visual effect, equipment that should be replaced in less than two years is indicated in red, equipment that should be replaced between 3-5 years is indicated in yellow, and equipment that should be replaced in more than five years is indicated in green, hence the name traffic light report. This report is shown with data relevant for the entire organization, and data relevant to each region in which the organization operates. For example, the report shows that at the initial time, in June 2020, the organization operated 13 Mill machines that should be replaced in less than 2 years. Conversely, at the end of the forecast period, in 2030, the organization would have only five MRI machines that should be replaced in less than two years.

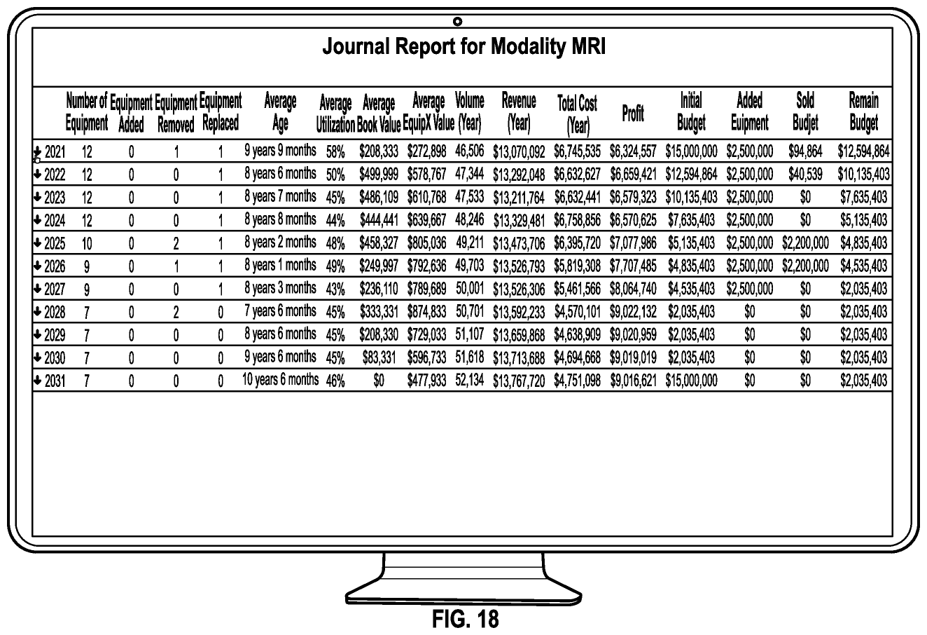

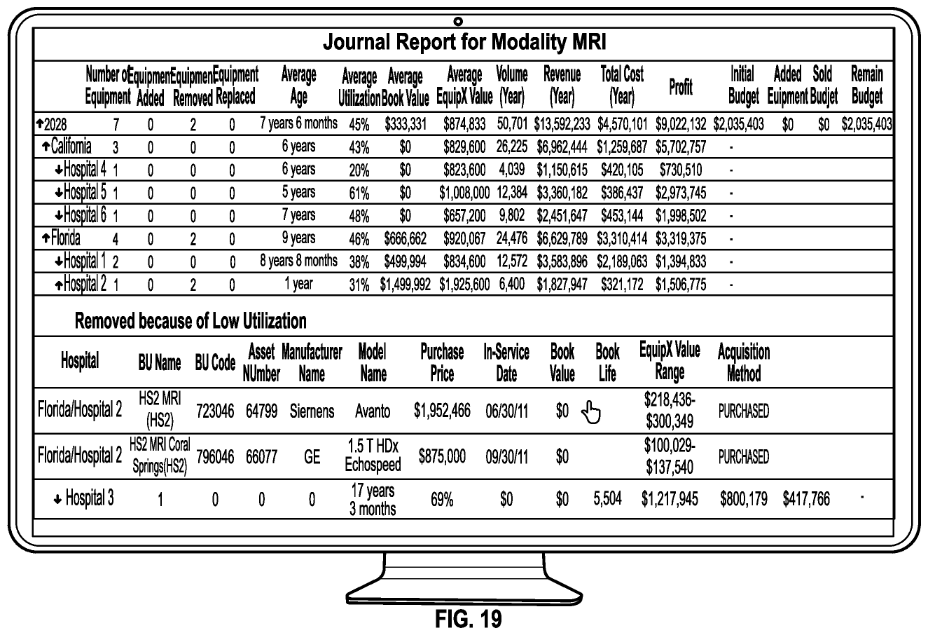

FIG. 18 illustrates a journal report. Many of the reports referred to herein may present a lot of data, such that it may not fit within the Figures. Therefore, the data may be shown as projected from the screen, or may not be shown altogether. As illustrated, the journal report is very detailed and provide relevant data of each year of the forecast. Moreover, the user may drill further by clicking links. For example, FIG. 19 illustrates part of the screen when the user clicks on 2028, at which point the data for each region is displayed, and the user further clicked on the region, at which point the data for each hospital in that region is shown. This can be done for each year and each region to view further detailed data.

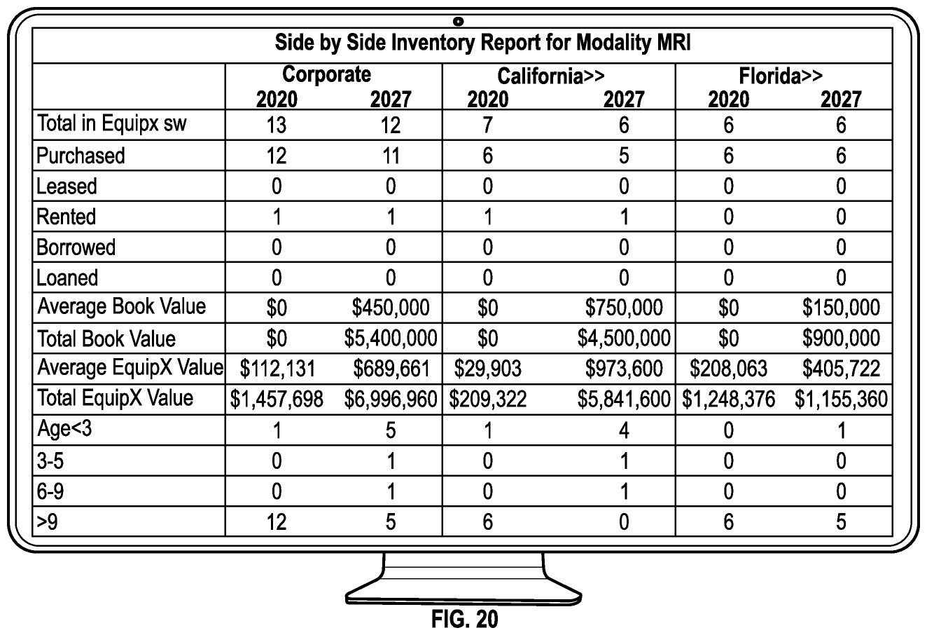

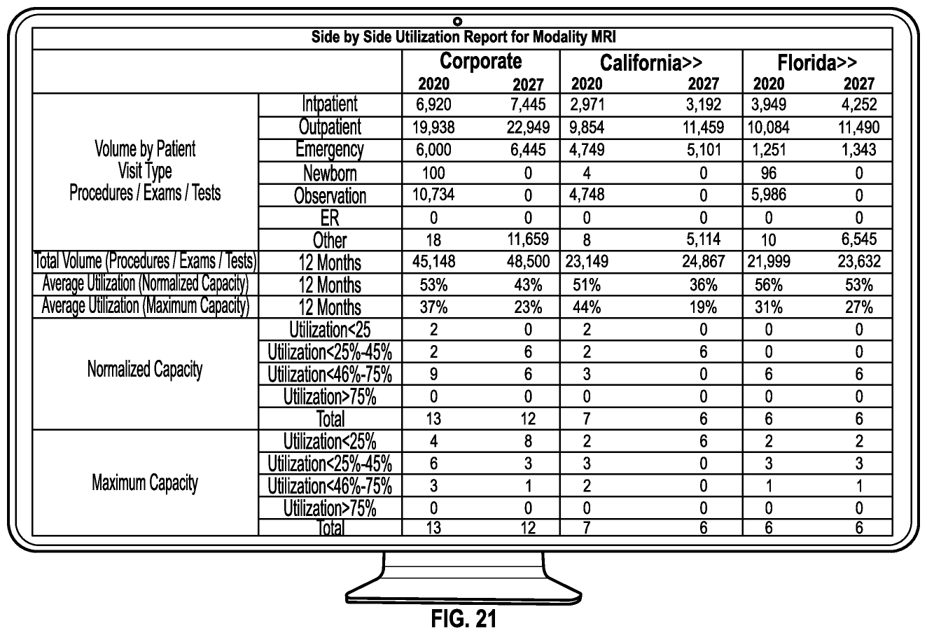

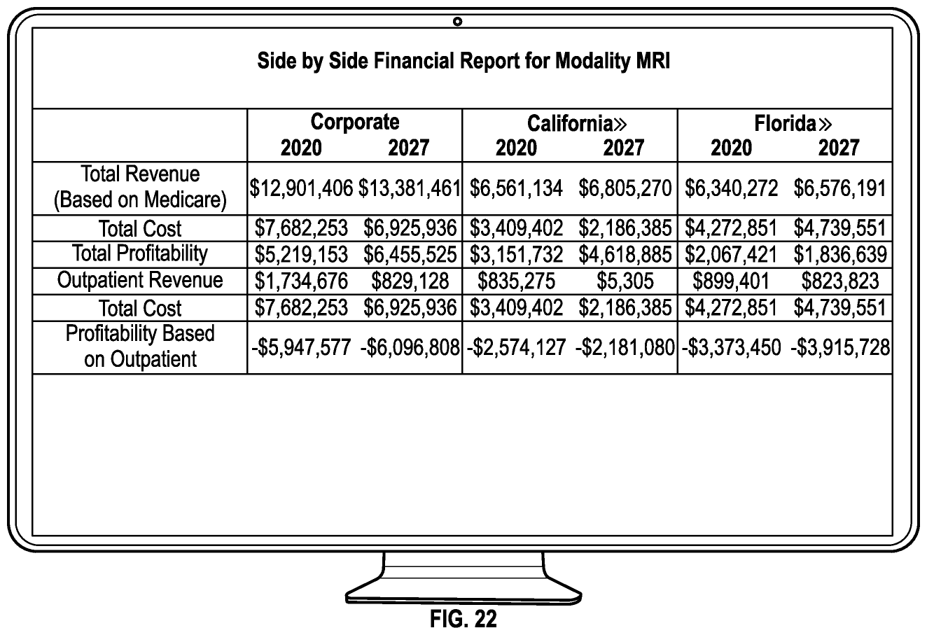

FIG. 20 illustrates a side by side report for the inventory at the initial date and at the forecast termination date. This report provides the data for the entire organization and separately for each region in which the organization operates. A more detailed information can be obtained by clicking on any of the regions, upon which the data for the corresponding hospitals or clinics will be displayed. Similarly, FIG. 21 illustrates a side by side report for the utilization at the initial date and at the forecast termination date. This report provides the data for the entire organization and separately for each region in which the organization operates. A more detailed information can be obtained by clicking on any of the regions, upon which the data for the corresponding hospitals or clinics will be displayed. FIG. 22 illustrates a side by side report for the financial status at the initial date and at the forecast termination date. This report provides the data for the entire organization and separately for each region in which the organization operates. A more detailed information can be obtained by clicking on any of the regions, upon which the data for the corresponding hospitals or clinics will be displayed.

An interesting entry in the financial report is the last line, showing profitability based on outpatients. For this entry, the profitability is calculated by subtracting the cost for all of the procedures performed from the revenue received only from outpatients. Of course, the equipment may be used for other than outpatients, such as inpatients, emergency room, observation, etc., for which the billing for the particular test or procedure is mixed with the total charge for the patient's overall treatment, and is therefore more difficult to separate. Thus, the profitability based on outpatients only provides an indication of the profitability of the equipment. That is, if the calculation results in a break even, it means that overall the equipment is profitable, i.e., the revenue from the outpatients covers the total expense of operating the machine, such that all other procedures are profitable.

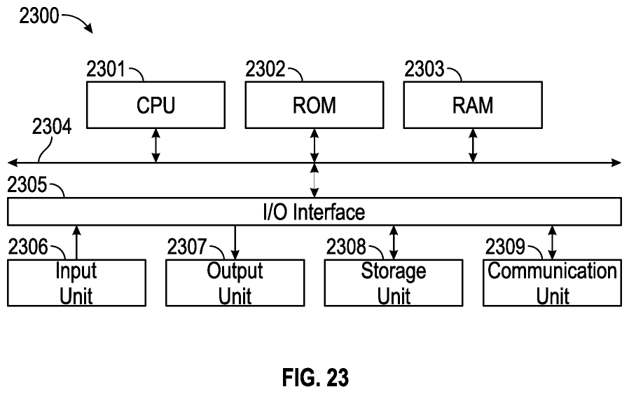

FIG. 23 schematically shows a block diagram of an apparatus 2300 for generating the forecasts according to example implementations of the present disclosure. As depicted, the apparatus 2300 includes a central process unit (CPU) 2301, which can execute various suitable actions and processing based on the computer program instructions stored in the read-only memory (ROM) 2302 or computer program instructions loaded in the random-access memory (RAM) 2303 from a storage unit 2308. The RAM 2303 can also store all kinds of programs and data required by the operations of the apparatus 2300. CPU 2301, ROM 2302 and RAM 2303 are connected to each other via a bus 2304. The input/output (I/O) interface 2305 is also connected to the bus 2304.

A plurality of components in the apparatus 2300 are connected to the I/O interface 2305, including: an input unit 2306, such as keyboard, mouse and the like; an output unit 2307, e.g., various kinds of display and loudspeakers etc.; a storage unit 2308, such as magnetic disk and optical disk etc.; and a communication unit 2309, such as network card, modem, wireless transceiver and the like. The communication unit 2309 allows the apparatus 2300 to exchange information/data with other devices via the computer network, such as Internet, and/or various telecommunication networks.

The above described modules and processes, such as the EquipX value calculation, the forecasting module, etc., can also be executed by the processing unit 2301. For example, in some implementations, the modules and processes can be implemented as a computer software program tangibly included in the machine-readable medium, e.g., the storage unit 2308. In some implementations, the computer program can be partially or fully loaded and/or mounted to the apparatus 2300 via ROM 2302 and/or the communication unit 2309. When the computer program is loaded to the RAM 2303 and executed by the CPU 2301, one or more steps of the above described modules, processes, or methods can be implemented. Alternatively, in other implementations, the CPU 2301 also can be configured in other suitable manners to realize the above modules, procedures and/or methods.

Thus, a system is disclosed for forecasting status for a fleet of medical equipment, the system comprising a processor and a memory storing executable instructions that, in response to execution by the processor, cause the system to at least: generate a user interface enabling a user to set a forecast period; obtain a list of existing equipment from a medical equipment database; operate a scoring module that assigns a score to each of the medical equipment for each year of the forecast period; calculate a market value for each of the equipment for each year of the forecast period; determine utilization rate for each of the medical equipment for each year of the forecast period; based on the score, the market value and the utilization rate, generate a replacement forecast by determining for each year of the forecast period whether any of the medical equipment should be replaced; and display the replacement forecast on the user interface.

The phrase “in one embodiment” is used repeatedly. The phrase generally does not refer to the same embodiment, however, it may. The terms “comprising”, “having” and “including” are synonymous, unless the context dictates otherwise. While the present invention has been related in terms of the foregoing embodiments, those skilled in the art will recognize that the invention is not limited to the embodiments described. The present invention may be practiced with modification and alteration within the spirit and scope of the appended claims. Thus, the description is to be regarded as illustrative instead of restrictive on the present invention.

While this invention has been discussed in terms of exemplary embodiments of specific materials, and specific steps, it should be understood by those skilled in the art that variations of these specific examples may be made and/or used and that such structures and methods will follow from the understanding imparted by the practices described and illustrated as well as the discussions of operations as to facilitate modifications that may be made without departing from the scope of the invention defined by the appended claims.

1. A system for forecasting status for a fleet of medical equipment, the system comprising a processor and a memory storing executable instructions that, in response to execution by the processor, cause the system to at least:
generate a user interface enabling a user to set a forecast period;
obtain a list of existing equipment from a medical equipment database;
operate a scoring module to assign a score to each of the medical equipment for each year of the forecast period;
calculate a market value for each of the equipment for each year of the forecast period;
determine utilization rate for each of the medical equipment for each year of the forecast period;
based on the score, the market value and the utilization rate, generate a replacement forecast by determining for each year of the forecast period whether any of the medical equipment should be replaced; and,
display the replacement forecast on the user interface.

2. The system of claim 1, wherein the scoring module assigns the score by, for each of the medical equipment, for each year of the forecast period:
assign points based on predicted operability of the medical equipment;
assign points based on predicted profitability of the medical equipment;
assign points based on the predicted technology refresh of the medical equipment; and,
summing all assigned points to generate the score.

3. The system of claim 2, wherein:
the user interface further enabling the user to set a minimum age; and,
at each year of the forecast period the scoring module assigns no points based on predicted operability to any of the medical equipment that is not older than the minimum age.

4. The system of claim 3, wherein the list of existing equipment includes end date for each of the medical equipment for which a manufacturer announced an end of life or end of support (EOL/EOS) date, and wherein the user interface further enables a user to enter point levels corresponding to the end date.

5. The system of claim 2, wherein the scoring module further awards points based on the ratio of the predicated market value to a predicated replacement cost.

6. The system of claim 2, wherein the points based on predicted profitability are determined for each year based on comparing profits and maintenance costs of each of the medical equipment for the year.

7. The system of claim 1, wherein the user interface enables a user to set a new equipment purchase price, depreciation period, utilization capacity, and operational cost.

8. The system of claim 7, wherein the utilization capacity comprises a maximum capacity and a normalized capacity, wherein the maximum capacity corresponds to capability of the medical equipment and the normalized capacity corresponds to potential utilization of the medical equipment.

9. The system of claim 1, wherein the user interface further enables the user to enter annual demographic change for the forecast period, and wherein the system uses the annual demographic change to determine the utilization rate.

10. The system of claim 2, wherein the user interface further enables the user to enter annual Medicare reimbursement change for the forecast period, and wherein the system uses the annual Medicare reimbursement change to determine the predicted profitability.

11. The system of claim 10, wherein the user interface further enables the user to enter annual operation cost change for the forecast period, and wherein the system uses the annual operational cost change to determine the predicted profitability.

12. The system of claim 1, wherein the replacement forecast displayed on the user interface comprises values at the initiation of the forecast and at the end of the forecast period for: number of medical equipment in the fleet, book value of the medical equipment, the market value for the medical equipment, utilization rate of the medical equipment, and total capital budget for implementing the forecast period.

13. The system of claim 1, wherein the system further produces a detailed report indicating for each year of the forecast period at least: number of new equipment acquired, amount expensed on the new equipment acquired, number of equipment sold, amount received for the equipment sold, utilization rate for each in service equipment, and age of each of the in service equipment.

14. A computer-readable storage medium for performing forecast of service equipment in a fleet, the computer-readable storage medium being non-transitory and having computer-readable program code stored therein that in response to execution by a processor, causes an apparatus to at least:
generate a user interface enabling a user to set a forecast period;
obtain operation data of the service equipment in the fleet;
form an appraisal module, the appraisal module returning an appraised value of each of the service equipment for each year in the forecast period;
form a scoring module that assigns a score to each of the service equipment for each year of the forecast period;
form a utilization forecast module to determine utilization rate for each of the service equipment for each year of the forecast period;
form a profitability forecast module to determine predicted profitability for each of the service equipment for each year of the forecast period;
form a decision support module that receives the score, the appraised value, the predicted profitability and the utilization rate, and generates an equipment replacement forecast by determining for each year of the forecast period whether any of the service equipment should be replaced; and,
display the equipment replacement forecast on the user interface.

15. The computer-readable storage medium of claim 14, wherein the scoring module assigns the score by, for each of the service equipment, for each year of the forecast period:
assign points based on predicted operability of the service equipment;
assign points based on the predicted profitability of the service equipment;
assign points based on the predicted technology refresh of the service equipment; and,
summing all assigned points to generate the score.

16. The computer-readable storage medium of claim 15, wherein the user interface further enabling the user to set a minimum age; and,
at each year of the forecast period the scoring module assigns no points based on predicted operability to any of the service equipment that is not older than the minimum age.

17. The computer-readable storage medium of claim 15, further comprising: forming a new price module providing new price forecast for new service equipment for each year of the forecast period; and wherein the scoring module assigns additional points according to a ratio of the appraised value to the new price forecast.

18. The computer-readable storage medium of claim 14, wherein the user interface further enables the user to enter annual demand change; and the utilization forecast module uses the annual demand change to determine utilization rate.

19. The computer-readable storage medium of claim 14, wherein the user interface further enables the user to enter equipment type; and wherein the equipment replacement forecast includes only service equipment corresponding to the equipment type.

20. The computer-readable storage medium of claim 15, wherein the equipment replacement forecast include information corresponding to corporate level, region level, and facility level.
